(19) United States
(12) Patent Application Publication
(10) Pub. No.: US 2024/0099781 A1
(43) Pub. Date: Mar. 28, 2024

(54) NEUROMAPPING SYSTEMS, METHODS, AND DEVICES

(71) Applicant: Medos International SARL, Le Locle (CH)

(72) Inventors: Marc Puls, Thorigen (CH); Eric Buehlmann, Duxbury, MA (US)

(21) Appl. No.: 18/371,712

(22) Filed: Sep. 22, 2023

Related U.S. Application Data
(60) Provisional application No. 63/410,794, filed on Sep. 28, 2022.

(51) Int. Cl. A61B 34/20; A61B 17/32; A61B 34/00; A61B 34/30; A61B 90/00

(52) U.S. Cl. CPC A61B 34/20; A61B 17/32; A61B 34/30; A61B 34/76; A61B 90/37; A61B 2017/00119; A61B 2034/2065; A61B 2090/064; A61B 2090/365

(57) ABSTRACT

Systems, methods, and devices are disclosed comprising a neuromonitoring probe, a navigation array attached to the probe, a tracking system to detect and track elements of the navigation array, and a controller having at least one processor configured to receive neuromonitoring data from the probe, receive probe positional data from the tracking system to determine a three-dimensional position and orientation of the probe, correlate the neuromonitoring data to the positional data to determine neuromapping data indicating a nerve position in the patient, and overlay a representation of the neuromapping data over three-dimensional position and orientation of the patient to establish a boundary around the nerve. The boundary may be a visual boundary on a display or a physical boundary in a computer-assisted surgical system.

### CROSS-REFERENCE TO RELATED APPLICATIONS

This application claims the benefit of U.S. Provisional Application Ser. No. 63/410,794, filed Sep. 28, 2022, the contents of which are incorporated by reference herein in their entirety.

### BACKGROUND

Detection of nerves in a patient is beneficial in a number of surgical procedures. Typically, neuromonitoring systems provide signals that indicate when a tip of a neuromonitoring probe is in proximity to a nerve. For example, the probe may use electromyography (EMG) to sense a voltage or a current. Alternatively, the probe may use mechanomyography (MMG) to sense an actual motion of a muscle associated with the nerve or the nerve itself. Moreover, neuromonitoring systems may operate in a variety of modes, depending upon whether a user (e.g., a surgeon) desires to determine direct contact with a nerve or a distance to a nerve.

However, it can be appreciated that the information resulting from neuromonitoring is relative as to the position of (e.g., distance from) the neuromonitoring probe. Once the neuromonitoring probe is moved, the user may remember generally where the nerve is located, but there is no positional data. Moreover, for surgical procedures where a user is working in close proximity to the nerve, it is important to know the nerve's exact location. Accordingly, there is a need for improved systems, methods, and devices to be used during a surgical procedure.

### SUMMARY

It is an important goal in the industry to integrate data from neuromonitoring systems into global coordinate systems, such as with reference to a patient's anatomy. This may be referred to as neuromapping. Neuromapping, for example, can be extremely useful in navigation, robotic, or augmented reality systems to visualize neural positions, such as on image data, when using surgical instruments with respect to a patient's anatomy.

Systems, methods, and devices are disclosed comprising a neuromonitoring probe, a navigation array attached to the probe, a tracking system to detect and track elements of the navigation array, and a controller having at least one processor configured to receive neuromonitoring data from the probe, receive positional data from the tracking system to determine a three-dimensional position and orientation of the probe, and correlate the neuromonitoring data to the positional data to determine neuromapping data indicating a nerve position in the patient.

Once the nerve position is determined, this neuromapping information can be used in a number of ways. For example, as will be described, a controller may overlay a representation of the neuromapping data (e.g., as an overlay image) over an image of the patient to define a boundary around the nerve, such as to allow a user (e.g., surgeon) to work in close proximity to the nerve while having a visual cue to avoid coming too close to the nerve.

In another example, a controller may define a boundary around the nerve and determine a series of control information to fence off the boundary to prevent computer-assisted surgical instruments from entering the boundary. For example, the controller may control a robotic arm so that a surgical instrument attached to the robotic arm avoids the boundary. Alternatively, the controller may be configured to provide haptic feedback when a tracked surgical instrument comes within a predefined proximity to the boundary.

### BRIEF DESCRIPTION OF THE DRAWINGS

FIG. 1 shows a schematic of a neuromapping system according to an embodiment of the present application.

FIG. 2 shows a schematic of neuromapping system according to another embodiment.

FIG. 3 shows a schematic of a neuromapping system architecture.

FIG. 4 shows a variety of examples of neuromapping displays associated with an endoscope or microscope.

FIG. 5 shows an example of a neuromapping display associated with an augmented reality (AR) device.

FIG. 6 shows a variety of examples of neuromapping displays associated with surgical planning.

FIG. 7 shows a robotic surgical system that may be modified to use neuromapping data.

### DETAILED DESCRIPTION

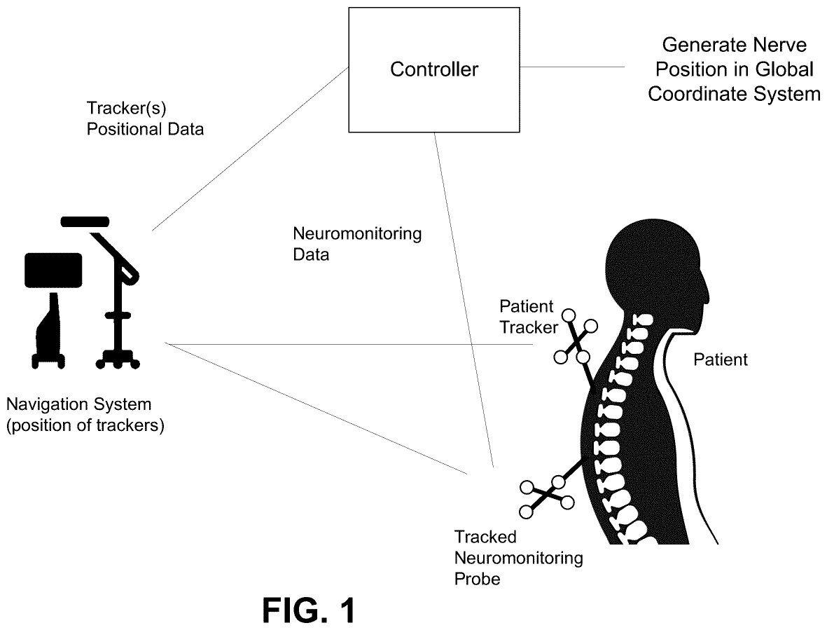

FIG. 1 shows a schematic of a neuromapping system. The system comprises a neuromonitoring probe. One example of a neuromonitoring probe is a SENTIO™ probe. The probe may use electromyography (EMG) to sense a voltage or a current associated, for example, with respect to a nerve's response to an electrical stimulus. The probe may use mechanomyography (MMG) to sense to actual motion of a muscle associated with the nerve or the nerve itself. The probe may operate in a direct contact mode as measured by sensors, for example, the probe may be configured to trigger a proximity alarm based on distance of the probe to the nerve. The probe may operate in a distance mode. For example, based on predetermined energy levels, a distance from the probe to the nerve may be determined. Neuromonitoring data may be sent to a controller or other information processing component. As may be appreciated, while input may be given (e.g., as to general location of the probe on the patient) so that the identity of the detected nerve may be determined, any positional information regarding this neuromonitoring data is relative to the probe and the nerve. Probes with multiple stimulators may be used to triangulate the position of the nerve.

To determine positional information of the nerve relative to the patient, surgical navigation or tracking may be employed. Examples of tracking systems in include optical tracking systems with reflective markers, radio frequency (RF) tracking systems, EMI tracking systems, visual systems including for example chest trackers, Aruco markers, machine vision using shape recognition, etc.

For example, optical navigation or tracking systems may utilize stereoscopic sensors to detect infra-red (IR) light reflected or emitted from one or more optical markers affixed to surgical instruments and/or portions of a patient's anatomy. A navigation array or tracker having a unique constellation or geometric arrangement of reflective elements may be coupled to a surgical instrument and, once detected by stereoscopic sensors, the relative arrangement of the elements in the sensors' field of view, in combination with the known geometric arrangement of the elements, may allow the system to determine a three-dimensional position and orientation of the tracker and, as a result, the instrument and/or anatomy to which the tracker is coupled.

Accordingly, a tracker may be mounted on the probe (e.g., integrally or removably). The tracker may be an optical tracker comprising reflective markers that may be detected by a navigation system. The tracker may comprise light emitting diodes (LEDs) as markers. The tracker may comprise RF trackers as markers. The tracker may comprise electromagnetic or EMI trackers as markers. The markers of the tracker may have a specific fixed geometric relationship such that they define a constellation, thereby indicating orientation of the probe. A distance and orientation between the probe tip and the tracker may be predetermined.

Optionally, a second tracker having a fixed geometric relationship may be coupled to a portion of patient anatomy, a surgical surface, or other immobile component. The second tracker may employ the same types of markers as the probe tracker. The second tracker may employ different types of markers as the probe tracker. The second tracker may represent a global coordinate system, for example, with reference to the patient. In some embodiments, the probe tracker is active (e.g., moving and detected at regular intervals) and the patient tracker is passive (e.g., immobile).

The navigation system and/or the controller may utilize the known fixed geometric relationship between the trackers to determine a precise three-dimensional position and orientation of the probe (and therefore the nerve). It is understood that in addition to the probe, the tracker may identify locations of additional instruments and devices.

The controller may be configured to correlate the neuromonitoring data and the tracker(s) positional data to determine a position of the nerve, for example, a three-dimension position of the nerve in the patient, thus providing neuromapping.

While the illustrated embodiments and accompanying description do not make particular reference to a specific surgery, the systems and methods described herein may be utilized in various applications involving robotic, robot-assisted, Augmented Reality (AR), Virtual Reality (VR), Mixed Reality (MR), and non-robotic systems for surgical approaches and/or operations where neuromapping may be appropriate. Example applications include knee surgery, e.g., total knee arthroplasty (TKA) or uni-compartmental knee arthroplasty (UKA), hip surgery, e.g., hip arthroplasty, shoulder surgery, spine surgery, etc. The teachings of the present disclosure may be applied to such procedures; however, the systems and methods described herein are not limited to these applications.

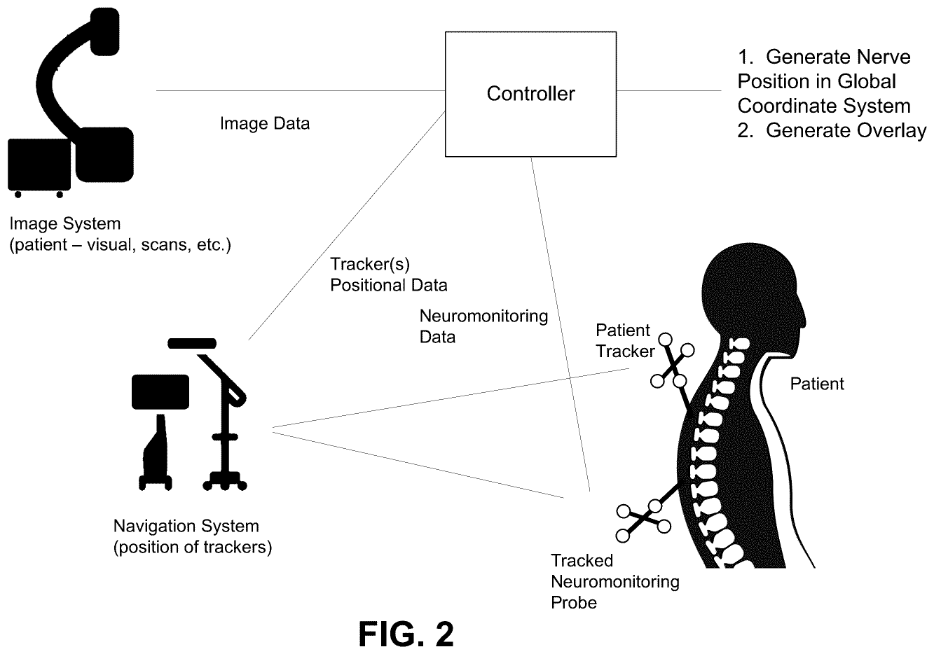

FIG. 2 shows a schematic of neuromapping system according to another embodiment. Aspects described with respect to FIG. 1 are understood to apply to this figure as well. Additionally, the controller receives image data, for example, from an image system. Examples of image data include real-time video, such as from a camera.

Examples of image data include stored images. The image data may comprise computed tomography (CT), magnetic resonance imaging (MRI), or other three-dimensional (3D) images. The image data may comprise unprocessed data or may be processed or segmented to show boundaries between different tissues (bone, nerve, muscle, etc.). The image data may comprise two-dimensional (2D) images, such as X-rays, or 2D images merged to afford a 3D image (e.g., fluoroscopy), or video images (e.g., from a TELIGEN™ camera, endoscopes, microscopes, etc.). The image data may be provided to the controller in a predetermined format, such as Digital Imaging and Communications in Medicine (DICOM) format.

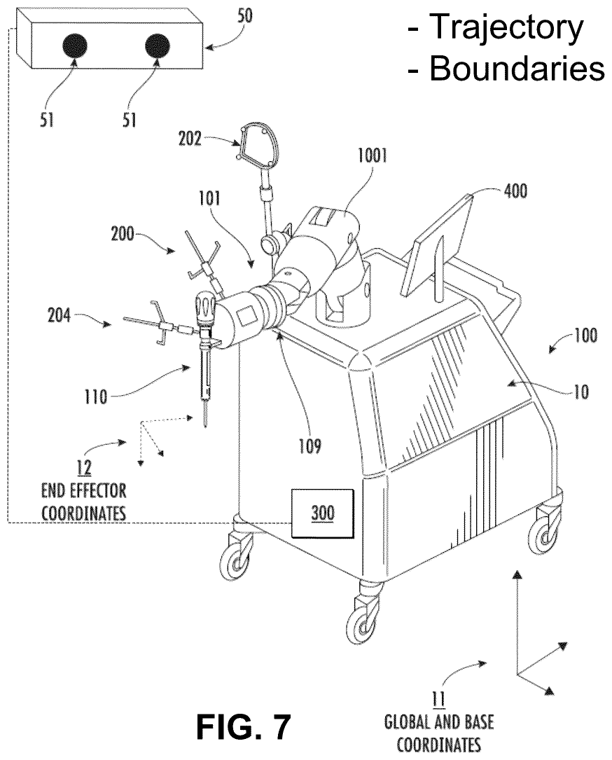

The controller may be configured to correlate the neuromonitoring data and the tracker(s) positional data to determine a position of the nerve, for example, a three-dimension position of the nerve in the patient, and then generate an overlay image representing a neuromapping display over the image data. The overlay may be visually perceptible by a user. Alternatively, as discussed with respect to FIG. 7, the overlay may be a boundary set around the determined position of the nerve.

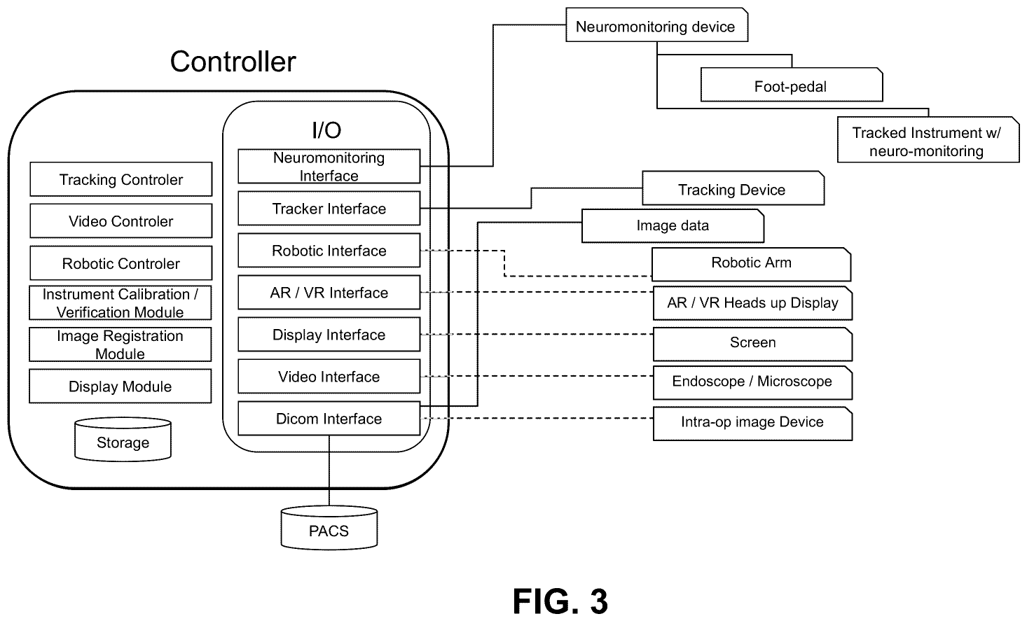

FIG. 3 shows a schematic of a neuromapping system architecture. The controller may receive input as described in FIG. 1 or FIG. 2 and may generate an overlay representing a neuromapping display over the image data. The overlay may be output to a device, for example, a display screen to be viewed by a surgeon, an Augmented Reality (AR), Mixed Reality (MR), or Virtual Reality (VR) heads-up display, an intra-op image device, an endoscope or microscope (or other video), or a robotic arm. The overlay image may be a stylized image, such as with a plurality of zones representing proximity to the nerve. Some examples are given below. The neuromonitoring device may be one or more of a neuromonitoring probe, a temperature probe, and a strain sensor.

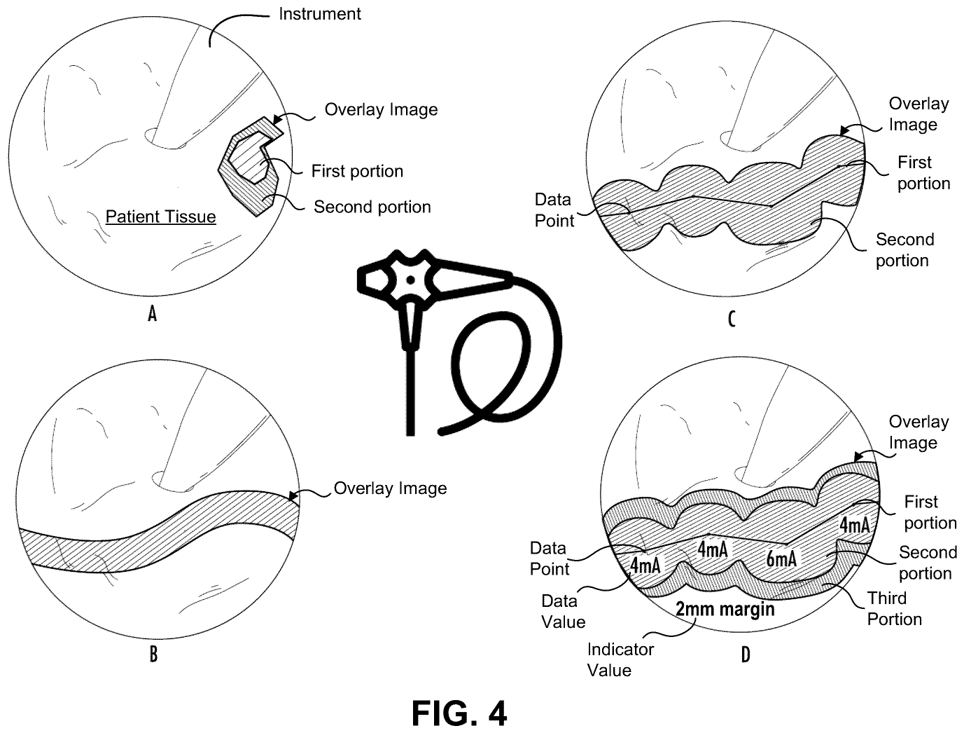

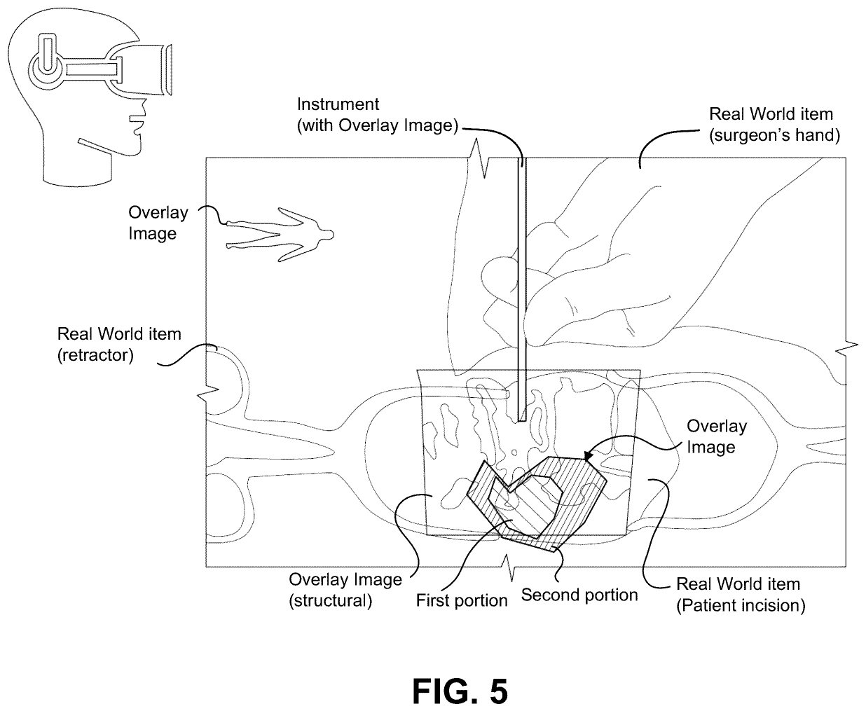

FIG. 4 shows a variety of examples of neuromapping displays associated with video of a surgical site. The video may be provided by a TELIGEN™ camera disposed in a cannula which provides a visual of tissue in spine surgery. The controller may receive image data from the camera. The video may also be associated with endoscopy systems and microscope systems or any surgical system. Alternatively, the video could be associated with an augmented reality (AR) device, as will be described in greater depth with respect to FIG. 5.

As an exemplary graphical user interface, at A, the video includes patient tissue and an instrument. A controller, such as previously described, may overlay an image representing the location of the nerve, superimposed on the video display. In this example, the overlay image comprises a plurality of zones around the nerve, e.g., based on the neuromapping data. Accordingly, a first portion of the overlay image may have a first appearance, for example, a first color (e.g., red), and a second, more distal portion of the overlay image may have a second appearance, for example, a second color (e.g., orange). The overlay image may serve as a warning to avoid critical portions of the nerve or may correspond to a distance to the nerve (e.g., to prevent damage by the instrument). The first and second colors may be used to convey degrees of warning to the surgeon.

In another example of video that includes patient tissue and an instrument, at B, the controller may overlay an image representing the location of the nerve, superimposed on the video display, in this example, the overlay image indicating a path of the nerve, e.g., based on the neuromapping data. The path of the nerve may be determined by the controller based on a best fit of the neuromonitoring data.

In another example, at C, the controller may overlay an image representing the location of the nerve, superimposed on the video display, in this example, the overlay image indicating a path of the nerve, e.g., based on the neuromapping data. The path of the nerve may be determined by the controller based on a best fit of the neuromonitoring data. Individual data points may be indicated as to location in the overlay image. A line (e.g., a first portion) connecting the points may be displayed to approximate the path of the nerve. A colored zone (e.g., a second portion) may be displayed around each point, for example, to indicate an amplitude of a neuromonitoring data. For example, stronger readings may have larger zones and/or different color schemes.

In another example, at D, the controller may overlay an image representing the location of the nerve, superimposed on the video display, in this example, the overlay image indicating a path of the nerve, e.g., based on the neuromapping data. The path of the nerve may be determined by the controller based on a best fit of the neuromonitoring data. Individual data points may be indicated as to location in the overlay image. A line (e.g., a first portion) connecting the points may be displayed to approximate the path of the nerve. A first colored zone (e.g., a second portion) may be displayed around each point, for example, to indicate an amplitude of a neuromonitoring data. For example, stronger readings may have larger zones. Specific readings or data values (e.g., for each individual point) may be displayed as part of the overlay. A second colored zone (e.g., a third portion) may be displayed around each point, for example, to indicate a margin of a predetermined width along the nerve path. An indicator value may be displayed, for example, to convey the magnitude of the margin in this example. The second portion and the third portions may be different colors and may be used to convey degrees of warning to the surgeon.

Alternatively, 3D segmented images may be enhanced with an overlay image to show areas where nerves have been confirmed by neuromapping. Various contemplated overlay images such as A-D are contemplated.

FIG. 5 shows an example of a neuromapping display associated with an augmented reality (AR) device. Examples of AR devices include a headset, a head mounted display (HMD), Google Glass, etc. The AR device includes an AR display which is configured to provide a real world scene to a user. The AR device has a point of view in providing the real world scene. The AR device has at least one processor and an integrated camera, for example, as part of the originally manufactured equipment.

A user (e.g., a surgeon) may view real world items through the device, which is to say through the camera of the AR device. For example, real world items in FIG. 5 include the patient (or at least the exterior of the patient and the incision), operating room equipment (such as drapes and retractors), the surgeon's hands, surgical instruments, etc.

The AR device is configured to also display overlaid virtual information (e.g., augmented reality information superimposed on the displayed real world scene). This overlaid virtual information may include stored information or streamed information, such as pictures, diagrams, navigational aids, video, text, warnings, models, simulations, etc. In particular, the overlaid virtual information may be image data (e.g., real-time video, stored images (2D and/or 3D patient images, unprocessed data or processed or segmented to show boundaries between different tissues (bone, nerve, muscle, etc.)). This overlaid virtual information (in this example, skeletal) may allow a surgeon to better visualize the patient anatomy than is possible from a real world view (even with magnification). For example, the instrument is a real world item, but a portion of the instrument may be inside the patient. Accordingly, the overlaid virtual information may represent the distal end of the instrument.

Using the neuromapping data, the controller (e.g., of the AR device or an associated controller) may also overlay an image representing the location of the nerve, superimposed on the video display of the AR device, in this example, the overlay image indicating a plurality of zones around the nerve, e.g., based on the neuromapping data. Accordingly, a first portion of the overlay image may have a first appearance, for example, a first color (e.g., red), and a second, more distal portion of the overlay image may have a second appearance, for example, a second color (e.g., orange). The overlay image may serve as a warning to avoid critical portions of the nerve or may correspond to a distance to the nerve (e.g., to prevent damage by the instrument). The first and second colors may be used to convey degrees of warning to the surgeon.

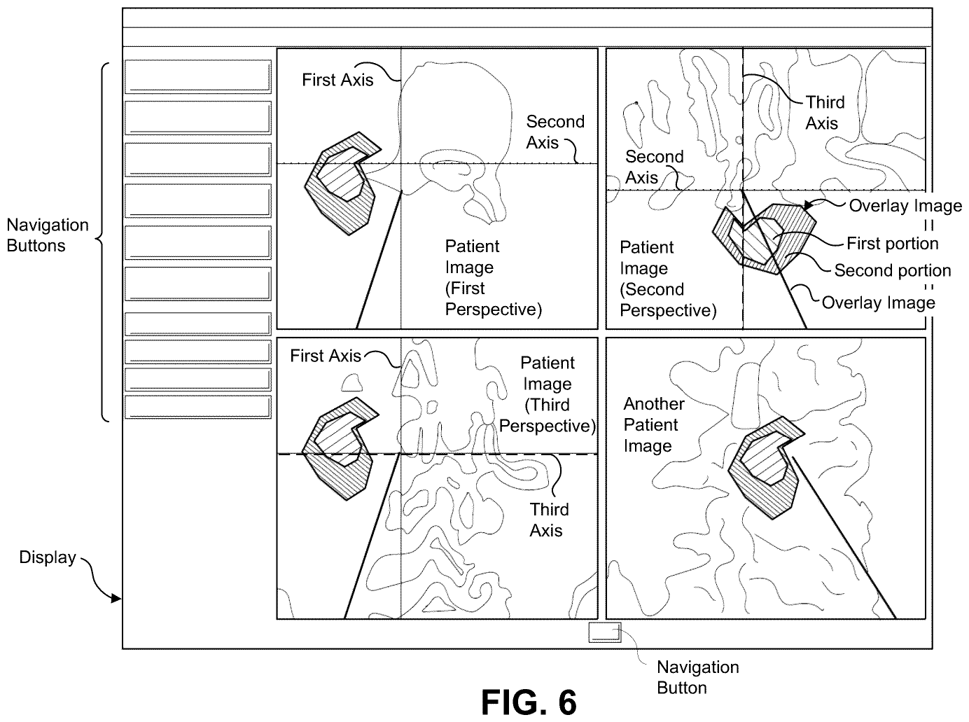

FIG. 6 shows a variety of examples of neuromapping displays associated with surgical planning. The surgery may be navigated surgery or may involve computer-assisted surgery. As illustrated, there are three displays using the same type of image data but with different perspectives. A fourth display embodies a different image data type. For example, the displays could be 2D and 3D image data, at least two different types of 2D image data, or at least two different types of 3D image data. The controller may overlay an image representing the location of the nerve, superimposed on a variety of views from different perspectives. In this example, the overlay image indicates a plurality of zones around the nerve, e.g., based on the neuromapping data. Accordingly, a first portion of the overlay image may be a first color (e.g., red), and a second, more distal portion of the overlay image may be a second color (e.g., orange). The overlay image may serve as a warning to avoid critical portions of the nerve (e.g., to prevent damage). The first and second colors may be used to convey degrees of urgency to the surgeon. The overlay image may be adjusted to account for the perspective based on the view to be superimposed upon. A surgeon may plan a trajectory (bold line in FIG. 6) with respect to a plurality of axes. The overlay image may assist the surgeon in planning a safe path for the trajectory to reach the destination. The system may suggest a safe path based on a combination of neuromapping data and other structures (organs, arteries, etc.) that represent no-go zones. In some embodiments, the controller may define a boundary around the nerve and determine a series of control information to fence off the boundary to prevent computer-assisted surgical instruments from entering the boundary. For example, the controller may control a robotic arm so that a surgical instrument attached to the robotic arm avoids the boundary. Alternatively, the controller may be configured to provide haptic feedback when a tracked surgical instrument (as described above) comes within a predefined proximity to the boundary.

FIG. 7 shows a schematic of a computer-assisted surgical system that may be modified to use neuromapping data, comprising a robotic device 100, including a surgical robot arm 1001, that includes an attached tool end effector 110 equipped with a cutting instrument (such as a scalpel, a saw, a drill, or a burr), and a plurality of arm segments 101 connected by rotatable or otherwise articulating joints 109. A distal-most segment of the robot arm may include a navigation array 200 mounted thereto adjacent to the tool end effector 110. As can be appreciated, positions of the end effector can be determined with respect to the patient or to the robotic device.

A global coordinate system 11 of the robotic device 100 may be defined, as well as an end effector coordinate system 12. The global coordinate system 11 may be defined in different ways and, in some embodiments, may use the location of a base 10 of the robotic device 110, which may or may not itself be stationary, as an origin. The location of the distal-most arm segment of the robotic device may be calculated by receiving a position signal from an encoder in each joint 109 and/or by measuring a position of the navigation array 200 to directly detect the position of the arm segment and determine the position of the distal end thereof in the global coordinate system. In some instances, a measured coordinate system of the navigation array 200 may be different from the global coordinate system 11 and calculations may be utilized to harmonize the two coordinate systems. In some embodiments, the measured coordinate system may be used as the global coordinate system 11. In some embodiments, the global coordinate system 11 may be a patient coordinate system.

The end effector coordinate system 12 may be defined in different ways but may refer to the position and orientation of the tool end effector 110 with respect to the operation of the tool end effector (e.g., if the tool end effector includes a cutting bit, the cutting direction may be along an “up” or “down” axis that might be defined by, e.g., a longitudinal axis of the tool). The tool end effector 110 held by the robotic device 100 may be constrained to move about the distal end of the distal-most arm segment such that the summation of the positions of joints 109 may define the location of the end effector coordinate system 12 in the global coordinate system 11 with respect to a control system of the joints 109 to control movement of the tool end effector 110.

Accordingly, the robotic device 100 may be connected to a controller 300 that controls, inter alia, the actuation of each joint 109 in order to position the tool end effector 110. The controller 300 typically comprises power supply, AC/DC converters, motion controllers, and other components to power the motors of the actuation units in each joint 109, as well as fuses, real-time control system interface circuits, and other components typically included in surgical robotic devices. Further, the present disclosure is also contemplated to include use of such instruments by surgical robots, by users with some degree of robotic assistance, and without involvement of surgical robots or robotic assistance (e.g., where solely surgical navigation/tracking is utilized).

Further, in some embodiments additional and/or alternative navigation arrays may be employed in addition to, or in place of, the navigation array 200 shown attached to a distal-most arm segment 101 of the robot arm 1001. For example, in some embodiments a navigation array 202 may be coupled to another component of the robotic device, such as a base of the robot arm 1001 in embodiments where the robot is mobile. Still further, a navigation array 204 may be coupled to the tool end effector itself. In embodiments where a single tool is provided, the array 204 may be coupled directly thereto.

A tracking unit 50 is provided, such that the relative pose or three-dimensional position and orientation of the navigation arrays 200, 202, and/or 204 (or other arrays) may be tracked in real time and shared to the controller 300 and any additional planning or control system. In some instances, coordinate systems may be attached to the robotic device 100 via the navigation array 200, the end effector 110 via the array 204, and an anatomical structure (not shown). The tracking unit 50 may measure the relative motions between any and all coordinate systems in real time. Real time may, in some embodiments, mean high frequencies greater than twenty Hertz, in some embodiments in the range of one hundred to five hundred Hertz, with low latency, in some embodiments less than five milliseconds. For example, the navigation arrays may include, for example, optical trackers comprising reflective or active markers detected by a sensor 51 in view of the surgical field. The tracking unit 50 may include a passive optical tracker consisting of, for example, a constellation of reflective tracking elements having a fixed geometric relationship that may be coupled to a portion of patient anatomy, a surgical instrument, or other component to be tracked. The tracking unit 50 may include a stereoscopic sensor having two or more physically separated detectors 51 that may be used to detect light reflected off each of the tracking elements (e.g., reflected infra-red (IR) light in some embodiments). The sensor 51, in some embodiments in conjunction with other information processing components such as the controller 300, may utilize the known fixed geometric relationship between the tracking elements and the detected positions of the tracking elements to determine a precise three-dimensional position and orientation of the navigation array(s), and therefore, of the entity coupled to the array.

In some embodiments, in place of, or in addition to, the above-described reflective optical tracking, optical tracking may be employed using active light emitters, such as light emitting diodes (LEDs). In other embodiments, electromagnetic trackers may be employed, while in still other embodiments any of inertial sensors using gyroscopic measurements, ultrasonic sensors, radio-frequency identification (RFID) sensors, or other known sensors may be employed.

The robotic arm may be controlled by the controller or may be moved by the surgeon. In either case, the controller may be configured to overlay a representation of the neuromapping data over three-dimensional position and orientation of the patient to establish a boundary around the nerve, and control or lock out the robotic arm to avoid the cutting instrument entering the boundary.

Alternatively, the controller may use the neuromapping data to guide the robot (e.g., to follow a determined trajectory) along a safe path (e.g., to avoid contacting the nerve) or safest path (if there are multiple paths to reach a target point). The determined trajectory may take other structures into account.

In yet another embodiment, the neuromonitoring data is temperature data. For example, temperature measurements can be used to ensure that certain cutting devices working with (or creating) heat do not destroy surrounding soft tissue (e.g., nerves). The overlay image could display temperature and the system may warn the user if predetermined thresholds are exceeded. In some embodiments, the neuromonitoring probe described above is replaced with a temperature probe (e.g., a tracked temperature probe). In some embodiments, temperature probe and the neuromonitoring probe described above are both part of the system.

In yet another embodiment, the neuromonitoring data is strain data. For example, strain measurements can be used to ensure that certain devices do not destroy surrounding soft tissue (e.g., nerves). Certain surgical tools, for example, retractors, exert pressure on the body tissue. Compressing or stretching a nerve for too long can cause nerve damage. Pressure sensors can be used to measure forces being applied by, for example, retractors. The overlay image could display strain on the nerve and the system may warn the user if predetermined thresholds are exceeded. In some embodiments, the neuromonitoring probe described above is replaced with a strain sensor probe (e.g., a tracked strain sensor). In some embodiments, strain sensor and the neuromonitoring probe described above are both part of the system.

In a first example, a surgical system is provided comprising a neuromonitoring probe, a navigation array attached to the probe, a tracking system to detect and track elements of the navigation array, and a controller having at least one processor configured to: receive neuromonitoring data from the probe, receive probe positional data from the tracking system to determine a three-dimensional position and orientation of the probe, correlate the neuromonitoring data to the positional data to determine neuromapping data indicating a nerve position in the patient, and overlay a representation of the neuromapping data (e.g., overlay image) over an image of the patient. The system may further comprise a display for displaying the overlay image. The display may be an augmented reality (AR) device. The system may further comprise a second navigation array affixed to the patient or a surgical surface. The image may be a live video feed. The live video feed may be from an endoscope and displayed on the AR device. The image may be a 2D image or a 3D image. The controller may be further configured to convey a warning if a predetermined threshold is exceeded. The controller may be further configured to receive temperature data from a temperature probe. The controller may be further configured to receive strain data from a pressure sensor.

In another example, a computer-assisted surgical system is provided comprising, a neuromonitoring probe, a navigation array attached to the probe, a tracking system to detect and track elements of the navigation array, an augmented reality (AR) device, a robotic arm having a cutting instrument, and a controller having at least one processor configured to: receive neuromonitoring data from the probe, receive probe positional data from the tracking system to determine a three-dimensional position and orientation of the probe, correlate the neuromonitoring data to the positional data to determine neuromapping data indicating a nerve position in the patient, overlay a representation of the neuromapping data over three-dimensional position and orientation of the patient to establish a boundary around the nerve, control the robotic arm to avoid the cutting instrument entering the boundary, and display the boundary on the AR device. The system may further comprise a second navigation array affixed to the patient or a surgical surface. The image may be a live video feed. The live video feed may be from an endoscope and displayed on the AR device. The image may be a 2D image or a 3D image. The controller may be further configured to convey a warning if a predetermined threshold is exceeded. The controller may be further configured to receive temperature data from a temperature probe. The controller may be further configured to receive strain data from a pressure sensor.

In yet another example, a computer-assisted surgical system is provided comprising an augmented reality (AR) device, a robotic arm having a cutting instrument, and a controller having at least one processor configured to: receive neuromapping data indicating a three-dimensional nerve position in a patient, overlay a representation of the neuromapping data over three-dimensional position and orientation of the patient to establish a boundary around the nerve, control the robotic arm to avoid the cutting instrument entering the boundary, and display the boundary on the AR device. The system may display the boundary as an overlay image based on the neuromapping data. The overlay image may comprise a plurality of zones around the nerve. The overlay image may comprise a first portion having a first appearance and a second portion having a second appearance, wherein the second portion is more distal with respect to the nerve. The overlay image may further comprise a third portion distal to the second portion to represent a margin. The controller may be further configured to provide haptic feedback to a user when the cutting instrument comes within a predefined proximity to the boundary.

In yet another example, a computer-assisted surgical system is provided comprising an augmented reality (AR) device and a controller having at least one processor configured to: receive neuromapping data indicating a three-dimensional nerve position in a patient, overlay a representation of the neuromapping data over three-dimensional position and orientation of the patient to establish a boundary around the nerve, and when a user of the AR device is looking at the nerve position, display the boundary on the AR device as an overlay image over the real world view based on the neuromapping data. The controller may be further configured to perform at least one of control a robotic arm having a cutting instrument to avoid the cutting instrument entering the boundary, or provide haptic feedback to the user of the AR device when the cutting instrument comes within a predefined proximity to the boundary. The overlay image may comprise a first portion having a first appearance and a second portion having a second appearance, wherein the second portion is more distal with respect to the nerve, for example, the first appearance and the second appearance may differ in color. The controller may be further configured to update the overlay image to account for a change in perspective of the user of the AR device.

1. A computer-assisted surgical system, comprising:
a neuromonitoring probe;
a navigation array attached to the probe;
a tracking system to detect and track elements of the navigation array;
an augmented reality (AR) device;
a robotic arm having a cutting instrument; and
a controller having at least one processor configured to:
receive neuromonitoring data from the probe;
receive probe positional data from the tracking system to determine a three-dimensional position and orientation of the probe;
correlate the neuromonitoring data to the positional data to determine neuromapping data indicating a nerve position in the patient;
overlay a representation of the neuromapping data over three-dimensional position and orientation of the patient to establish a boundary around the nerve;
control the robotic arm to avoid the cutting instrument entering the boundary; and
display the boundary on the AR device.

2. The system of claim 1, further comprising a second navigation array affixed to the patient or a surgical surface.

3. The system of claim 1, wherein the image is a live video feed.

4. The system of claim 1, wherein the live video feed is from an endoscope.

5. The system of claim 1, wherein the image is a 3D image.

6. The system of claim 1, wherein the image is a 2D image.

7. The system of claim 1, wherein the controller is further configured to convey a warning if a predetermined threshold is exceeded.

8. The system of claim 1, wherein the controller is further configured to receive temperature data from a temperature probe.

9. The system of claim 1, wherein the controller is further configured to receive strain data from a pressure sensor.

10. A computer-assisted surgical system, comprising:
an augmented reality (AR) device;
a robotic arm having a cutting instrument; and
a controller having at least one processor configured to:
receive neuromapping data indicating a three-dimensional nerve position in a patient;
overlay a representation of the neuromapping data over three-dimensional position and orientation of the patient to establish a boundary around the nerve;
control the robotic arm to avoid the cutting instrument entering the boundary; and
display the boundary on the AR device.

11. The system of claim 10, wherein the boundary is displayed as an overlay image based on the neuromapping data.

12. The system of claim 11, wherein the overlay image comprises a plurality of zones around the nerve.

13. The system of claim 12, wherein the overlay image comprises a first portion having a first appearance and a second portion having a second appearance, wherein the second portion is more distal with respect to the nerve.

14. The system of claim 13, wherein the overlay image further comprises a third portion distal to the second portion to represent a margin.

15. The system of claim 10, wherein the controller is further configured to provide haptic feedback to a user when the cutting instrument comes within a predefined proximity to the boundary.

16. A computer-assisted surgical system, comprising:
an augmented reality (AR) device;
a robotic arm having a cutting instrument; and
a controller having at least one processor configured to:
receive neuromapping data indicating a three-dimensional nerve position in a patient;
overlay a representation of the neuromapping data over three-dimensional position and orientation of the patient to establish a boundary around the nerve; and
when a user of the AR device is looking at the nerve position, display the boundary on the AR device as an overlay image over the real world view based on the neuromapping data.

17. The system of claim 16, wherein the controller is further configured to perform at least one of:
control the robotic arm to avoid the cutting instrument entering the boundary; or
provide haptic feedback to the user of the AR device when the cutting instrument comes within a predefined proximity to the boundary.

18. The system of claim 16, wherein the overlay image comprises a first portion having a first appearance and a second portion having a second appearance, wherein the second portion is more distal with respect to the nerve.

19. The system of claim 18, wherein the first appearance and the second appearance differ in color.

20. The system of claim 16, wherein the controller is further configured to update the overlay image to account for a change in perspective of the user of the AR device.
